(19) United States
(12) Patent Application Publication
(10) Pub. No.: US 2007/0100146 A1
(43) Pub. Date: May 3, 2007

(54) Process for the preparation of imidazo[4,5-c]-quinolin-4-amines

(76) Inventor: Trevor Dzwiniel, Madison, WI (US)

Correspondence Address: Richard J. Hammond, Ph.D., 5218 Riverbend Blvd., Baton Rouge, LA, 70820, US

(21) Appl. No.: 11/266,701

(22) Filed: Nov. 3, 2005

(51) Int. Cl. C07D 471/02

(52) U.S. Cl. 546/82

(57) ABSTRACT

The present invention is a method for preparing a compound of the formula 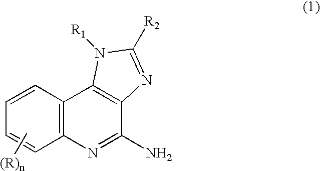
wherein R is hydrogen, C₁ to C₆ alkyl, C₁ to C₆ alkoxy or halo, R₁ and R₂ are independently hydrogen, C₁ to C₁₀ alkyl, C₁ to C₁₀ alkoxy, C₃ to C₁₀ cycloalkyl, C₃ to C₁₀ alkenyl, C₅ to C₁₀ cycloalkenyl, C₂ to C₁₀ alkynyl, C₆ to C₂₀ aryl or substituted C₆ to C₂₀ aryl and n is the integer 1 or 2 by reacting the corresponding N-oxide with an aqueous solution of ammonia and a C₁ to C₆ alkyl, C₆ to C₂₀ aryl or substituted C₆ to C₂₀ aryl chloroformate thereby forming said compound formula (1).

### BACKGROUND OF THE INVENTION

1. Field of the Invention

This invention relates to a process for preparing 1-H-imidazo[4,4-c]quinolines. More particularly, this invention relates to processes for the preparation of 1-substituted-1H-imidazo[4,5-c]quinolin-4-amines.

2. Description of the Related Art

The compounds of formula (1) are known to have physiological activity, particularly as antiviral agents or as intermediates for the preparation of such agents. For example, the compound where R₁ is isobutyl and R and R₂ are hydrogen, i.e., 1-(2-methylpropyl)-1H-imidazo[4,5-c]quinolin-4-amine, is a well-known ingredient in compositions useful for the treatment of genital warts in humans.

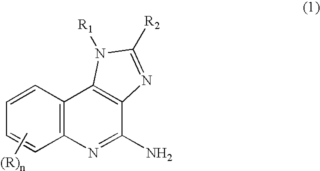

The synthesis of the compounds of formula (1) has been described in numerous patents. In U.S. Pat. No. 4,689,338 (Gerster) the ammonolysis of the corresponding 4-chloro compounds is disclosed. The compounds formed include those where R₁ is alkyl, hydroxylalkyl and the like, R is alkyl, alkoxy, or halo and R₂ is hydrogen, alkyl, aryl, etc. The reaction is carried out under pressure, e.g., in a sealed reactor, at temperatures up to about 155° C. for about 16-18 hours. Also see U.S. Pat. No. 4,929,624 (Gerster et al) disclosing the preparation of similar compounds, including those where R₁ is alkenyl, by the sealed reactor ammonolysis reaction. The publication Shen et al., Chem. Res. & Appl., 2001, 13, 249-252 reports that the process of U.S. Pat. No. 4,689,339 may be carried out at the somewhat lower temperature of 100° C. and for a shorter time, e.g., 4 hours.

The above method used to prepare the compounds of formula (1) are particularly disadvantageous since they must be performed in a high pressure reactor at elevated temperature.

An additional method has been disclosed to prepare to compounds of formula (1) in U.S. Pat. No. 6,069,149 (Namba et al). The method set forth in this patent is a two step process that starts with a compound that is a precursor to the compounds of formula (1). Such compound bears a 4-chloro substituent rather than an amino group and identifies the groups R as hydrogen, R₁ as a series of 2 to 12 methylene groups terminated with a diamine and R₂ as hydrogen, alkyl substituted with aryl, aryloxy or alkoxy and phenyl. In the first reaction, the '149 patent discloses that the 4-chloro precursor compound undergoes an addition\elimination reaction, which is accomplished by heating it with benzylamine in an appropriate solvent or with an excess of benzylamine without a solvent. The 4-benzylamino substitution product is obtained. The second step is the hydrogenation or hydrogenolysis of the 4-benzylamine analog. According to this patent, the 4-benzylamino compound is heated with a carboxylic acid (preferably formic acid) in the presence of a palladium hydroxide\carbon catalyst for 1 to 2 days to provide the compounds of formula (1). However, in another reaction, under acidic conditions, and with hydrogen in the presence of palladium on carbon, the 4-benzylamino analog failed to react. See Shen et al above.

The above method used to prepare the compounds of formula (1) of the '149 patent is particularly disadvantageous since the reaction solvent must be removed in the first step before any further reaction. Additionally, the hydrogenolysis requires an extended reaction period and produces the product in only very low yields, i.e., about 40%.

U.S. Patent Application 2005/0085500 (Gutman et al) discloses another method for preparing compounds of formula (1). In this application, a three step reaction is employed where the final step is the basic hydrolysis of the acid addition salts of the compounds of formula (1). The acid addition salts are produced by hydrolysis of a 4-(arylmethyl)amino-1H-imidazo(4,5-c)quinoline with a strong acid, e.g., sulfuric acid, methanesulfonic acid, etc. The patent application discloses that the 4-(arylmethyl)amino-1H-imidazo(4,5-c)quinoline can be prepared by the reaction of an arylmethylamine with the 4-chloro analog of the compounds of formula (1), e.g., by reaction with benzylamine.

The above patent application suffers many of the disadvantages of the previously discussed prior art patents, i.e., relatively low yields, long reaction times, difficulty in purifying reaction products and the like. Thus, there is a continuing need for improving the methods for the preparation of 4-amino-1H-imidazo(4,5-c)quinolines.

### SUMMARY OF THE INVENTION

The present invention is a method for preparing a compound of the formula 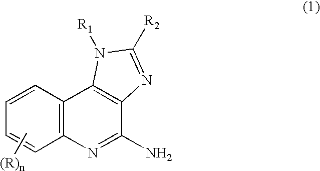
wherein R is hydrogen, C₁ to C₆ alkyl, C₁ to C₆ alkoxy or halo, R₁ and R₂ are independently hydrogen, C₁ to C₁₀ alkyl, C₁ to C₁₀ alkoxy, C₃ to C₁₀ cycloalkyl, C₃ to C₁₀ alkenyl, C₅ to C₁₀ cycloalkenyl, C₂ to C₁₀ alkynyl, C₆ to C₂₀ aryl or substituted C₆ to C₂₀ aryl and n is the integer 1 or 2. The method comprises reacting an N-oxide of formula (4) 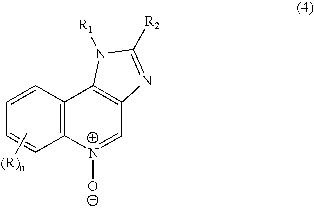
where R, R₁ and R₂ are as previously defined, with an aminating agent and a C₁ to C₆ alkyl, C₆ to C₂₀ aryl or substituted C₆ to C₂₀ aryl chloroformate thereby forming said compound formula (1).

### DESCRIPTION OF THE PREFERRED EMBODIMENTS

For the purpose of the present invention, the following definitions apply:

The term “C₁ to C₆ alkyl” and\or “C₁ to C₁₀ alkyl” is intended to mean a straight or branched chain having from 1 to 6 carbon atoms or 1 to 10 carbon atoms respectively, such as exemplified by the groups methyl, ethyl, n-propyl, isopropyl, n-butyl, isobutyl, n-pentyl, 2-methylpentyl, n-hexyl, 2-methylhexyl and the like.

The term “C₁ to C₆ alkoxy” and\or “C₁ to C₁₀ alkoxy” is intended to mean a straight or branched chain having from 1 to 6 carbon atoms or 1 to 10 carbon atoms attached to an oxygen atom such as exemplified methoxy, ethoxy, n-propoxy, isopropoxy, n-butoxy, isobutoxy, n-pentoxy, and the like.

The term “C₃ to C₁₀ cycloalkyl” is intended to mean a cyclic group having at least 3 carbon atoms in such cyclic group such exemplified by cyclopropyl, cyclobutyl, cyclopentyl, cyclohexyl, methylcyclopropyl, methylcyclobutyl, ethylcyclopentyl, ethylcyclohexyl and the like.

The term “C₃ to C₁₀ alkenyl” is intended to mean a straight or branched chain having from 3 to 10 carbon atoms with a double bond between at least two of such carbon atoms such exemplified by propenyl. 2-butenyl, 2-pentenyl, 3-pentenyl, 1,3-pentadiene and the like.

The term “C₅ to C₁₀ cycloalkenyl” is intended to mean a cyclic group having at least 5 carbon atoms in such cyclic group and additionally containing at least one double bond between two of the carbon atoms in the cyclic group such exemplified by cyclopentene, cyclohexene, cycloheptene, 1,3-cyclohexadiene and the like.

The term “C₂ to C₁₀ alkynyl” is intended to mean a straight or branched chain having from 2 to 10 carbon atoms in such chain and additionally containing at least one triple bond such as acetylenyl, propynyl, 1-butynyl, 1,3-butadiynyl and the like.

The term “C₆ to C₂₀ aryl or substituted C₆ to C₂₀ aryl” is intended to mean the aromatic hydrocarbon groups having at least 6 carbon atoms in the aromatic ring and optionally bearing at least one substituent that may include C₁ to C₆ alkyl, C₁ to C₆ alkoxy, C₃ to C₁₀ cycloalkyl, halo, C₆ to C₂₀ aryl or substituted C₆ to C₂₀ aryl and the like as defined above such exemplified by phenyl, naphthyl, 2-chlorophenyl, 3-methylphenyl and the like.

The term “halo” is intended to mean at least one fluoro, chloro, bromo or iodo.

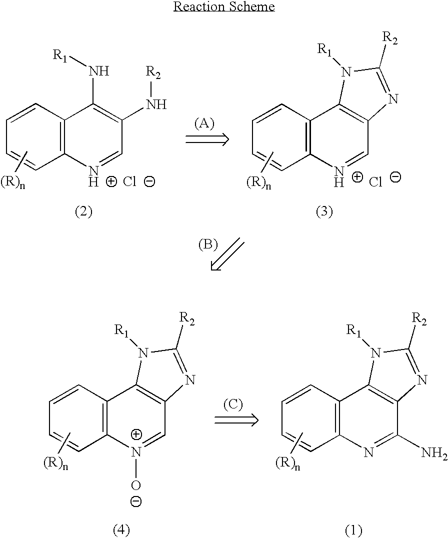

One embodiment of the present invention is illustrated by the Reaction Scheme shown above wherein R is hydrogen, C₁ to C₆ alkyl, C₁ to C₆ alkoxy or halo, R₁ and R₂ are independently hydrogen, C₁ to C₁₀ alkyl, C₁ to C₁₀ alkoxy, C₃ to C₁₀ cycloalkyl, C₃ to C₁₀ alkenyl, C₅ to C₁₀ cycloalkenyl, C₂ to C₁₀ alkynyl, C₆ to C₂₀ aryl or substituted C₆ to C₂₀ aryl and n is the integer 1 or 2.

The above embodiment involves the preparation of 1H-imidazo[4,5-c]quinolin-5-ium chloride optionally substituted at the 1 and 2 positions with C₁ to C₁₀ alkyl, C₁ to C₁₀ alkoxy , C₃ to C₁₀ cycloalkyl, C₃ to C₁₀ alkenyl, C₅ to C₁₀ cycloalkenyl, C₂ to C₁₀ alkynyl, C₆ to C₂₀ aryl or substituted C₆ to C₂₀ aryl and optionally substituted at positions 6, 7, 8 or 9 with C₁ to C₆ alkyl, C₁ to C₆ alkoxy or halo, Intermediate (3), by reaction of 3,4-diamino or 3 amino and 4 amino each substituted with C₁ to C₁₀ alkyl, C₁ to C₁₀ alkoxy, C₃ to C₁₀ cycloalkyl, C₃ to C₁₀ alkenyl, C₅ to C₁₀ cycloalkenyl, C₂ to C₁₀ alkynyl, C₆ to C₂₀ aryl or substituted C₆ to C₂₀ aryl quinolin, Intermediate (2),with a tri C₁ to C₁₀ alkyl orthoformate. Preferably the reaction is carried with a 3-amino-4-C₁ to C₁₀ alkyl aminoquinoline hydrochloride, most preferably with 3-amino4-isobutylaminoquinoline hydrochloride to produce 1-C₁ to C₁₀alkyl-1H-imidazo[4,5-c]quinolin-5-ium chloride or 1-isobutyl-1H-imidazo[4,5-c]quinolin-5-ium chloride respectively. See Reaction Step (A). The reaction is carried out in an inert solvent, e.g., a C₁ to C₁₀ alkyl halide employing a tri C₁ to C₆ alkyl orthoformate most preferably a triethyl orthoformate or trimethyl orthoformate at from about 40° to about 100° C., preferably from about 70° to about 95° C., most preferably from about 80° to about 90° C. The most preferred solvent is dichloromethane.

The product of the above reaction, Intermediate (3), is removed from the reaction mixture by any conventional means (for example, by filtration) and dried, e.g., air-drying, vacuum drying, etc., before further use.

To a solution of Intermediate (3) is added an aqueous solution of a base (to neutralize the acid and convert it to the free base). Such free base then is the reacted with an oxidizing agent such as hydrogen peroxide or an organic peroxide to yield the N-oxide, Intermediate (4) where n, R, R₁ and R₂ are as defined above. See Reaction Step (B). The solvent for the Intermediate (3) solution is one that is inert to the reaction and may be the same as that used in the first reaction disclosed above.

Any organic or inorganic base may be used in the above reaction step to prepare Intermediate (4). Preferably such base is inorganic base. The preferred base is selected from the group consisting of the alkali metal or the alkaline earth metal hydroxides, carbonates or bicarbonates, most preferably sodium bicarbonate.

The oxidation of Intermediate (3) is carried out with an oxidizing agent. Such oxidizing agents are illustrated by hydrogen peroxide or an organic peroxide. The organic peroxides include peroxyacids, acyl peroxides, diacyl peroxides and are illustrated by peracetic acid, peroxyhexanoic acid, β-phenylperacetic acid, cyclohexane-peroxycarboxylic acid, diperoxyhexanedioic acid, etc. The reaction is typically conducted at room temperature. Intermediate (4), a 1H-imidazo[4,5-c]quinolin-5-oxide, is a crystalline solid. It can be removed from the reaction solution by any conventional means, such as by removing the solvent (e.g., by distillation) and subsequent crystallization from the remaining liquid. The oxidizing agent most preferably used in Reaction Step (B) is peracetic acid.

Intermediate (4), dissolved in an inert solvent, is reacted with an aminating agent, e.g., ammonia gas, conc. ammonium hydroxide (an aqueous solution of ammonia), ammonium salts (ammonium carbonate, ammonium phosphate, etc.) and the like. The resulting reaction solution is then treated with a C₁ to C₆ alkyl, C₆ to C₂₀ aryl or substituted C₆ to C₂₀ aryl chloroformate. See Reaction Step (C). The chloroformate is added to the reaction solution of Intermediate (4) and the aminating agent at such a rate that the reaction solution temperature does not exceed about 15° C., preferably 10° C., most preferably 0° C. Typically the reaction temperature is in the range of from about −15° C. to about 15° C. A 4-amino-1H-imidazo(4,5-c)quinoline of formula (1) forms, typically as a solid, in the ensuing reaction mass. It can be readily removed by, for example, filtration, decantation, etc.

It is preferred that the Reaction Step (C) is carried out using a C₁ to C₆ alkyl chloroformate, most preferably with ethyl chloroformate and the solvent for such reaction is a C₁ to C₆ halocarbon, such a dichloromethane.

Any suitable method may be used to purify the 4-amino-1H-imidazo(4,5-c)quinolines of formula (1), Suitable purification methods include, but are not limited to, slurrying, crystallizing and chromatography. Suitable solvents for slurrying and crystallizing include dichloromethane, DMF and the like. Additional methods of purification are well known to those skilled in the art.

The present invention provides a method for preparing 4-amino-1H-imidazo(4,5-c)quinolines of formula (1) in high purity and high yields from the corresponding N-oxides of formula (4). The present invention, being carried out at atmospheric pressure, is safe and easily useful for industrial application. It is illustrated, but not limited by the following examples.

### EXAMPLES

### Example 1

### (a)

A 1 liter, 3-necked round bottom flask equipped with a mechanical stirrer was charged with the hydrochloride salt of 3-amino-4-isobutylaminoquinoline (328.2 g, 1304 mmol). The yellow solid was slurried in triethyl orthoformate (1200 mL) and heated to 85±5° C. for 7 hours. After cooling, the colorless slurry was filtered and washed with methyl tert-butyl ether (MTBE) (2×300 mL) and air-dried. The resulting solid was further dried under vacuum for several hours. The intermediate, 1-isobutyl-1H-imidazo[4,5-c]quinolin-5-ium chloride (384.5 g) was obtained (112%).

### (b)

The intermediate from the above reaction, 1-isobutyl-1H-imidazo[4,5-c]quinolin-5-ium chloride (206 g, 787 mmol) was slurried in methylene chloride (1200 mL) and treated portion wise with a saturated solution of sodium bicarbonate (1 L). There was significant gas evolution and the material dissolved to give a two phase, slightly yellow solution. The layers were separated and the organic layer was treated with peracetic acid (215 mL). The two phase system was stirred overnight. The dichloromethane layer was separated and washed with water and cautiously with sodium bicarbonate until pH 7 was reached. The dichloromethane solution was then concentrated to about one half the volume. Ethyl acetate (equivolume to the reduced solution) was added and the solution was further concentrated. During this period, the product began to precipitate. The solution was distilled until no more water was evident in the distillate. The resulting suspension was filtered and the solid N-oxide was washed with ethyl acetate and air-dried to afford an off-white solid. A back extraction of the combined aqueous layers, when worked-up similarly, gave a smaller batch of N-oxide. The combined yield of this intermediate, 1-isobutyl-1H-imidazo[4,5-c]quinoline-5-oxide was 150.7 g, 79%.

### (c)

The intermediate 1-isobutyl-1H-imidazo[4,5-c]quinoline-5-oxide prepared from the above reaction (64.6 g, 268 mmol) was dissolved in dichloromethane (640 mL) and cooled to about 10° C. in a methanol ice-bath. Ammonium hydroxide (100 mL was added. The two-phase system was then treated with a solution of ethyl chloroformate (36 mL, 400 mmol) in dichloromethane (75 mL). The solution was added at a rate to keep the temperature below 0° C. After the addition was complete, the thick suspension was allowed to warm to room temperature for one hour. This thick slurry was filtered through a D-sized sintered glass frit. The solid recovered from the frit was washed with water (2×200 mL), methanol (3×150 mL) and air dried to yield about 100 g. The solid was recrystallized from dimethylformamide (DMF) (1300 mL) at 140° C. and washed with ethanol. Imiquimod (1-isobutyl-1H-[4,5-c]quinolin-4-ylamine) was isolated as a white solid, 48.6 g, 76% yield, HPLC: 99.6.

The Imiquimod from the above reaction was combined with another batch, the combination having a total weight about 66 g, and was dissolved in acidic ethanol (300 mL 1M HCl and 400 mL EtOH) at 55° C. To this solution was added conc. ammonium hydroxide (75 mL) and water (200 mL). A thick white precipitate formed immediately upon addition. The mixture was cooled to room temperature, filtered and washed with ethanol (200 mL), water (200 mL) and ethanol (200 mL), The white solid was air-dried, then placed in a vacuum oven overnight to yield 66.06 g.

I claim:

1. A method for preparing a compound of the formula 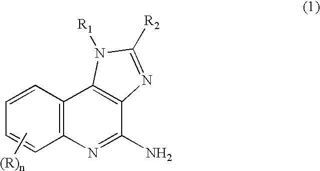
wherein R is hydrogen, C₁ to C₆ alkyl, C₁ to C₆ alkoxy or halo, R₁ and R₂ are independently hydrogen, C₁ to C₁₀ alkyl, C₁ to C₁₀ alkoxy, C₃ to C₁₀ cycloalkyl, C₃ to C₁₀ alkenyl, C₅ to C₁₀ cycloalkenyl, C₁ to C₁₀ alkynyl, C₆ to C₂₀ aryl or substituted C₆ to C₂₀ aryl comprising reacting an N-oxide of the formula 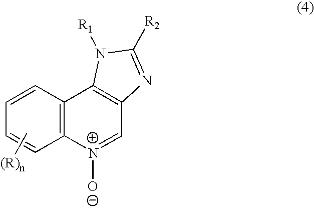
where R, R₁ and R₂ are as previously defined, with an aminating agent and a C₁ to C₆ alkyl, C₆ to C₂₀ aryl or substituted C₆ to C₂₀ aryl chloroformate thereby forming said compound of formula (1).

2. The method according to claim 1 wherein the reaction is carried out at a temperature from about −15° to about 15°.

3. The method according to claim 1 wherein said aminating agent is ammonia and the chloroformate is a C₁ to C₆ alky chloroformate.

4. The method according to claim 1 wherein said chloroformate is ethyl chloroformate.

5. The method according to claim 1 wherein said N-oxide is prepared by reacting an acid addition salt of the formula 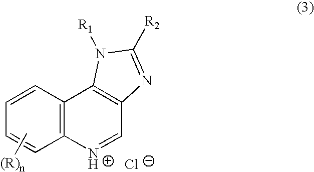
where R, R₁ and R₂ are as previously defined, with a base to neutralize said acid addition salt and then treating said neutralized acid addition salt with an oxidizing agent to provide said N-oxide.

6. The method according to claim 3 wherein said base is an inorganic base selected from the group consisting of the alkali metal or the alkaline earth metal hydroxides, carbonates or bicarbonates.

7. The method according to claim 3 wherein said oxidizing agent is a peroxy acid, acyl peroxide or diacyl peroxide.

8. The method according to claim 3 wherein said oxidizing agent is peracetic acid.

9. The method according to claim 3 wherein said acid addition salt is prepared by treating a quinoline salt of the formula 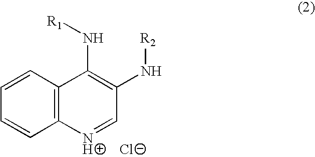
with a tri(C₁ to C₆ alkyl) orthoformate.

10. The method according to claim 9 wherein said orthoformate is triethyl orthoformate or trimethyl orthoformate.

11. A method for preparing a compound of the formula 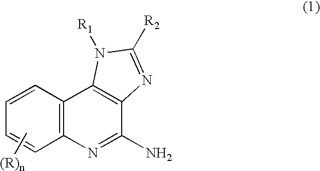
wherein R is hydrogen, C₁ to C₆ alkyl, C₁ to C₆ alkoxy or halo, R₁ and R₂ are independently hydrogen, C₁ to C₁₀ alkyl, C₁ to C₁₀ alkoxy, C₃ to C₁₀ cycloalkyl, C₃ to C₁₀ alkenyl, C₅ to C₁₀ cycloalkenyl, C₁ to C₁₀ alkynyl, C₆ to C₂₀ aryl or substituted C₆ to C₂₀ aryl comprising
a) treating a quinoline salt of the formula 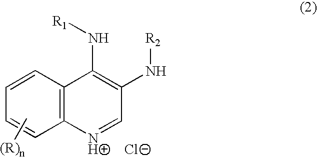
where R, R₁ and R₂ are as previously defined, with a tri(C₁ to C₆ alkyl) orthoformate to yield a compound of formula (3). 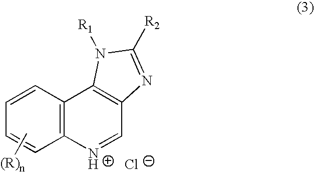
where R, R₁ and R₂ are as previously defined,
b) reacting the compound of formula (3) with a base and then with an oxidizing agent to provide a compound of formula (4) 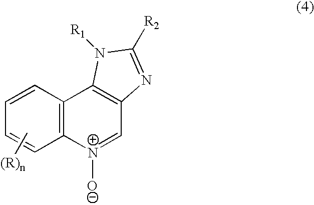
where R, R₁ and R₂ are as previously defined,
c) reacting the compound of formula (4) with an aminating agent and a C₁ to C₆ alkyl, C₆ to C₂₀ aryl or substituted C₆ to C₂₀ aryl chloroformate thereby forming said compound of formula (1).
